(19) United States
(12) Patent Application Publication
(10) Pub. No.: US 2012/0021461 A1
(43) Pub. Date: Jan. 26, 2012

(54) ISOTHERMAL STRAND DISPLACEMENT AMPLIFICATION

(75) Inventors: Douglas Spencer Millar, Revesby NSW (AU); Claire Kate Inman, Artarmon NSW (AU)

(73) Assignee: HUMAN GENETIC SIGNATURES PTY. LTD., North Ryde, NSW (AU)

(21) Appl. No.: 13/144,274

(22) PCT Filed: Jan. 15, 2010
(86) PCT No.: PCT/AU2010/000055

§ 371 Date: Oct. 14, 2011

(30) Foreign Application Priority Data
Jan. 21, 2009 (AU) 2009900222

(51) Int. Cl. C12P 19/34

(52) U.S. Cl. 435/91.2

(57) ABSTRACT

A method for isothermal DNA amplification comprising: providing to the DNA to be amplified an amplification mix comprising a first primer at least partially complementary to a region of DNA and containing Xanthosine, a second primer at least partially complementary to a region of DNA and containing Xanthosine, a DNA polymerase, an enzyme capable of strand displacement, an enzyme that recognises Xanthosine in double-stranded DNA and causes a nick or excises a base in one DNA strand at or near Xanthosine; and amplifying the DNA substantially without thermal cycling.

### TECHNICAL FIELD

The present invention relates to improved methods for amplifying nucleic acid molecules substantially without thermal cycling.

### BACKGROUND ART

The most widely used method for amplification of specific sequences from within a population of nucleic acid sequences is that of polymerase chain reaction (PCR) (Dieffenbach C and Dveksler G eds. PCR Primer: A Laboratory Manual. Cold Spring Harbor Press, Plainview N.Y.). In this amplification method, oligonucleotides, generally 15 to 30 nucleotides in length on complementary strands and at either end of the region to be amplified, are used to prime DNA synthesis on denatured single-stranded DNA templates. Successive cycles of denaturation, primer hybridisation and DNA strand synthesis using thermostable DNA polymerases allows exponential amplification of the sequences between the primers. RNA sequences can be amplified by first copying using reverse transcriptase to produce a cDNA copy. Amplified DNA fragments can be detected by a variety of means including gel electrophoresis, hybridisation with labeled probes, use of tagged primers that allow subsequent identification (eg. by an enzyme linked assay), use of fluorescently-tagged primers that give, rise to a signal upon hybridisation with the target DNA (eg. Beacon and TaqMan systems).

One disadvantage of PCR is the need of a thermocycler to heat and cool the amplification mixture to denature the DNA. This, amplification cannot be carried out in primitive sites or operated easily outside of a laboratory environment.

As well as PCR, a variety of other techniques have been developed for detection and amplification of specific sequences. One example is the ligase chain reaction (Barany F Genetic disease detection and DNA amplification using cloned thermostable ligase. Proc. Natl. Acad. Sci. USA 88:189-193, 1991).

In addition to conventional methods of DNA amplification that rely on the thermal denaturation of the target during the amplification reaction, a number of methods have been described that do not require template denaturation during the amplification reaction and are thus termed isothermal amplification technologies.

Isothermal amplification was first described in 1992 (Walker G T, Little M C, Nadeau J G and Shank D. Isothermal in vitro amplification of DNA by a restriction enzyme/DNA polymerase system. PNAS 89: 392-396, 1992) and termed Strand Displacement Amplification (SDA). Since then, a number of other isothermal amplification technologies have been described including Transcription Mediated Amplification (TMA) and Nucleic Acid Sequence Based Amplification (NASBA) that use an RNA polymerase to copy RNA sequences but not corresponding genomic DNA (Guatelli J C, Whitfield K M, Kwoh D Y, Barringer K J, Richmann D D and Gingeras T R. Isothermal, in vitro amplification of nucleic acids by a multienzyme reaction modeled after retroviral replication. PNAS 87: 1874-1878, 1990; Kievits T, van Gemen B, van Strijp D, Schukkink R, Dircks M, Adriaanse H, Malek L, Sooknanan R, Lens P. NASBA isothermal enzymatic in vitro nucleic acid amplification optimized for the diagnosis of HIV-1 infection. J Virol Methods. 1991 December; 35(3):273-86).

Other DNA-based isothermal techniques include Rolling Circle Amplification (RCA) in which a DNA polymerase extends a primer directed to a circular template (Fire A and Xu S Q. Rolling replication of short circles. PNAS 92: 4641-4645, 1995), Ramification Amplification (RAM) that uses a circular probe for target detection (Zhang W, Cohenford M, Lentrichia B, Isenberg H D, Simson E, Li H, Yi J, Zhang D Y. Detection of Chlamydia trachomatis by isothermal ramification amplification method: a feasibility study. J Clin Microbiol. 2002 January; 40(1):128-32) and more recently, Helicase-Dependent isothermal DNA amplification (HDA), that uses a helicase enzyme to unwind the DNA strands instead of heat (Vincent M, Xu Y, Kong H. Helicase-dependent isothermal DNA amplification. EMBO Rep. 2004 August; 5(8):795-800).

Traditional amplification techniques rely on continuing cycles of denaturation and renaturation of the target molecules at each cycle of the amplification reaction. Heat treatment of DNA results in a certain degree of shearing of DNA molecules, thus when DNA is limiting such as in the isolation of DNA from a small number of cells from a developing blastocyst, or particularly in cases when the DNA is already in a fragmented form, such as in tissue sections, paraffin blocks and ancient DNA samples, this heating-cooling cycle could further damage the DNA and result in loss of amplification signals. Isothermal methods do not rely on the continuing denaturation of the template DNA to produce single stranded molecules to serve as templates from further amplification, but rely on enzymatic nicking of DNA molecules by specific restriction endonucleases at a constant temperature.

The technique termed Strand Displacement Amplification (SDA) relies on the ability of certain restriction enzymes to nick the unmodified strand of hemi-modified DNA and the ability of a 5′-3′ exonuclease-deficient polymerase to extend and displace the downstream strand. Exponential amplification is then achieved by coupling sense and antisense reactions in which strand displacement from the sense reaction serves as a template for the antisense reaction (Walker G T, 1992). Such techniques have been used for the successful amplification of Mycobacterium tuberculosis (Walker G T, 1992), HIV-1, Hepatitis C and HPV-16 Nuovo G J, 2000), Chlamydia trachomatis (Spears P A, Linn P, Woodard D L and Walker G T. Simultaneous Strand Displacement Amplification and Fluorescence Polarization Detection of Chlamydia trachomatis. Anal. Biochem. 247: 130-137, 1997).

The use of SDA to date has depended on modified phosphorthioate nucleotides in order to produce a hemi-phosphorthioate DNA duplex that on the modified strand would be resistant to enzyme cleavage, resulting in enzymic nicking instead of digestion to drive the displacement reaction. Recently, however, several “nickase” enzyme have been engineered. These enzymes do not cut DNA in the traditional manner but produce a nick on one of the DNA strands. “Nickase” enzymes include N.Alw1 (Xu Y, Lunnen K D and Kong H. Engineering a nicking endonuclease N.Alw1 by domain swapping. PNAS 98: 12990-12995, 2001), N.BstNB1 (Morgan R D, Calvet C, Demeter M, Agra R, Kong H. Characterization of the specific DNA nicking activity of restriction endonuclease N.BstNBI. Biol. Chem. 2000 November 381(11):1123-5) and Mly1 (Besnier C E, Kong H. Converting Mlyl endonuclease into a nicking enzyme by changing its oligomerization state. EMBO Rep. 2001 September; 2(9):782-6. Epub 2001 Aug. 23).

In addition, SDA has been improved by the use of a combination of a heat stable restriction enzyme (Ava1) and Heat stable Exo-polymerase (Bst polymerase). This combination has been shown to increase amplification efficiency of the reaction from a 10⁸ fold amplification to 10¹⁰ fold amplification so that it is possible using this technique to amplify unique single copy molecules. The resultant amplification factor using the heat stable polymerase/enzyme combination is in the order of 10⁹ (Milla M. A., Spears P. A, Pearson R E and Walker G T. Use of the Restriction Enzyme Ava1 and Exo-Bst Polymerase in Strand Displacement Amplification Biotechniques 24:392-396, 1997).

To date, all isothermal DNA amplification techniques require the initial double stranded template DNA molecule to be denatured prior to the initiation of amplification. In addition, amplification is only initiated once from each priming event.

Non-regular DNA bases such as inosine, deoxyinosine, 8 deoxyguanine, hydroxyuracil, 5-methyl-dC, 5 hydroxyuridine, 5 bromo-dU inosine with C, ribonucleotides, and uracil have been found to be useful in isothermal amplification, WO 2006/125267 (Human Genetic Signatures Pty Ltd).

A new non-regular base has now been found by the present inventor to perform between 10 and 1000 fold better than the preferred prior art non-regular base, inosine, in isothermal amplification.

The present inventor has developed an improved amplification method which utilises a non-regular base, enzymes and primers, and which method does not require repeated temperature cycling.

### DISCLOSURE OF INVENTION

In a first aspect, the present invention provides a method for isothermal DNA amplification comprising:

providing to DNA to be amplified an amplification mix comprising:
a first primer at least partially complementary to a region of DNA and containing Xanthosine,
a second primer at least partially complementary to a region of DNA and containing Xanthosine,
a DNA polymerase, an enzyme capable of strand displacement,
an enzyme that recognises Xanthosine in double-stranded DNA and causes a nick or excises a base in one DNA strand at or near the Xanthosine; and

amplifying the DNA substantially without thermal cycling.

Optionally, the DNA can be denatured prior to, during, or after addition of the amplification mix.

Preferably, the first primer is at least partially complementary to a region of a first strand of DNA, and the second primer is at least partially complementary to a region of DNA of the second strand of DNA.

Xanthosine:

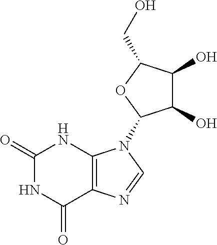

The first and second primers can be oligonucleotides, oligonucleotide analogues, oligonucleotides of chimeric nature such as PNA/oligonucleotides or INA/oligonucleotides. Preferably, the primers are deoxyoligonucleotides.

Preferably, the oligonucleotide analogue is selected from intercalating nucleic acid (INA), peptide nucleic acid (PNA), hexitol nucleic acid (HNA), MNA, altritol nucleic acid (ANA), locked nucleic acid (LNA), cyclohexanyl nucleic acid (CAN), CeNA, TNA, (2′-NH)-TNA, nucleic acid based conjugates, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R—RNA, 2′-OR—RNA, α-L-RNA, and β-D-RNA, and mixtures thereof and hybrids thereof, as well as phosphorous atom modifications thereof, such as but not limited to phosphorothioates, methyl phospholates, phosphoramidites, phosphorodithiates, phosphoroselenoates, phosphotriesters and phosphoboranoates. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

By INA is meant an intercalating nucleic acid in accordance with the teaching of WO 03/051901, WO 03/052132, WO 03/052133 and WO 03/052134 (Unest A/S, assigned to Human Genetic Signatures Pty Ltd) incorporated herein by reference. An INA is an oligonucleotide or oligonucleotide analogue comprising one or more intercalator pseudonucleotide (IPN) molecules.

When a primer having Xanthosine binds to DNA it forms a site recognised by the enzyme.

The primers can have one or more Xanthosines. In some situations, two or more Xanthosines can improve the amplification process. The Xanthosines can be positioned close or spaced apart by at least several regular bases.

The DNA polymerase can be any suitable polymerase such as Taq polymerase Stoffel fragment, Taq polymerase, Advantage DNA polymerase, AmpliTaq, Amplitaq Gold, Titanium Taq polymerase, KlenTaq DNA polymerase, Platinum Taq polymerase, Accuprime Taq polymerase, Pfu polymerase, Pfu polymerase turbo, Vent polymerase, Vent exo-polymerase, Pwo polymerase, 9° Nₘ DNA polymerase, Therminator, Pfx DNA polymerase, Expand DNA polymerase, rTth DNA polymerase, DyNAzyme™ EXT Polymerase (an optimized mixture of DyNAzyme II DNA Polymerase and a proofreading enzyme. DyNAzyme II DNA Polymerase is isolated and purified from an E. coli strain expressing the cloned DyNAzyme DNA Polymerase gene from Thermus brockianus, New England Biolabs Inc, USA), Klenow fragment, DNA polymerase 1, DNA polymerase, T7 polymerase, Sequenase™ (a genetically engineered form of T7 DNA polymerase which retains polymerase activity with virtually no 3′-5′ exonuclease activity, Affymetrix Inc, USA), Tfi polymerase, T4 DNA polymerase, Bst polymerase, Bca polymerase, phi-29 DNA polymerase and DNA polymerase Beta or modified versions thereof.

The strand displacement enzyme can be any suitable enzyme such as polymerases, helicases, AP endonucleases, mismatch repair enzymes capable of stand displacement or genetically (or otherwise) modified enzyme capable of stand displacement.

In a preferred form, the DNA polymerase also has strand displacement capability. The DNA polymerase can be any suitable polymerase having strand displacement capability. Examples include, but not limited to, Klenow exo- (New England Biolabs (NEB) catalogue number M0212S), Bst DNA polymerase large fragment (NEB catalogue number M0275S), Vent exo- (NEB catalogue number M0257S), Deep Vent exo- (NEB catalogue number M0259S), M-MuLV reverse transcriptase (NEB catalogue number M0253S), 9° Nm DNA polymerase (NEB catalogue number M0260S) and Phi29 DNA polymerase (NEB catalogue number M0269S) ThermoPhi™ (Prokaria ehf), Tfi polymerase (Invitrogen) and BCa polymerase (Takara). Preferably, the DNA polymerase is either Klenow Exo- or Bst polymerase.

Preferably, the DNA polymerase is exonuclease deficient.

The enzyme can be any suitable enzyme that is capable of recognising Xanthosine in double stranded DNA and can cause a nick or excise a base at or near the site of the Xanthosine.

Preferably, the enzyme is Endonuclease V, hOGG1 or Fpg. In a particularly preferred embodiment the enzyme is Endonuclease V. In another preferred embodiment the enzyme is Fpg.

It will be appreciated that other suitable enzymes can be made or obtained that recognise Xanthosine in double stranded DNA and act as required by nicking or causing base removal in the method according to the present invention.

The additives required for DNA amplification include nucleotides, buffers or diluents such as magnesium or manganese ions, co-factors, etc known to the art.

The amplification mix can also contain nucleotides, buffers or diluents such as magnesium or manganese ions, co-factors and suitable additives such as single stranded binding proteins such as T4gp32, RecA or SSB.

Amplification can be carried out at any suitable temperature where the enzymes have desired activity. Typically, the temperature can be about 20° C. to about 75° C., about 25° C. to 60° C. For the enzymes used in the current study, about 42° C. has been found to be particularly suitable, especially when using the mesophilic Klenow exo-enzyme and 60° C. using the thermostable Bst polymerase. It will be appreciated that other temperatures, either higher or lower, can be used and would include ambient or room temperature. Importantly, the present invention does not require thermal cycling to amplify nucleic acids.

In a preferred embodiment, the amplification mix further includes salt (NaCl) to improve amplification reaction. For the enzyme heat stable Bst polymerase/TMA Endonuclease V combination up to about 100 mM NaCl was found to improve amplification. About 50 mM NaCl was found to be preferred.

In one preferred from, the DNA is pre-treated with a modifying agent which modifies cytosine bases but does not modify 5′-methyl-cytosine bases under conditions to form single stranded modified DNA. Preferably, the modifying agent is selected from bisulphite, acetate or citrate and treatment does not result in substantial DNA fragmentation. More preferably, the agent is sodium bisulphite, a reagent, which in the presence of water, modifies cytosine into uracil.

Sodium bisulphite (NaHSO₃) reacts readily with the 5,6-double bond of cytosine to form a sulfonated cytosine reaction intermediate which is susceptible to deamination, and in the presence of water gives rise to a uracil sulfite. If necessary, the sulfite group can be removed under mild alkaline conditions, resulting in the formation of uracil. Thus, potentially all cytosines will be converted to uracils. Any methylated cytosines, however, cannot be converted by the modifying reagent due to protection by methylation.

Preferred methods for bisulphite treatment of nucleic acid can be found in WO 2004/096825 in the name of Human Genetic Signatures Pty Ltd (Australia), incorporated herein by reference.

If both strands of the treated DNA need to be amplified in the same amplification reaction, then four primers can be used (ie two primers for each of the modified strands of DNA).

In a second aspect, the present invention provides a primer for isothermal DNA amplification containing at least one internal Xanthosine and when bound to a region of DNA forms a site recognised by an enzyme capable of causing a nick or excising a base in one DNA strand at or near the site of the Xanthosine.

In a third aspect, the present invention provides use of a primer according to the second aspect of the present invention for DNA amplification substantially without thermal cycling.

The amplification method of the present invention can be used as a replacement for PCR or other known DNA amplification processes. Uses include, but not limited to, detection of disease, amplifying desired genes or segments of DNA or RNA, SNP detection, real time amplification procedures, amplifying bisulphite treated DNA, whole genome amplification methods, adjunct to cloning methods, in situ amplification of DNA on cytological specimens, such as detection of microbes in sections or smears, detection of microbes in food contamination, amplification of breakpoints in chromosomes such as BCR-ABL translocations in various cancers, amplification of sequences inserted into chromosomes that may be oncogenic and predictive of disease progression, such as HPV fragment insertion, detection of methylated versus unmethylated sequences in normal versus cancerous cells, and in in situ tests for methylation changes in IVF tests for the normalcy of blastocyst development and the amplification and detection of infectious agents.

A distinct advantage of the present invention is that it can be carried out directly on double stranded DNA. The invention can also used for RNA by carrying out reverse transcription of the RNA prior to isothermal amplification. Furthermore, the present invention does not require heating or cooling for amplification. It is contemplated that the method according to the present invention can be carried ‘in the field’ i.e. at room or ambient temperature without the need for powered laboratory equipment.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed prior to development of the present invention.

In order that the present invention may be more clearly understood, preferred embodiments will be described with reference to the following drawings and examples.

### DESCRIPTION OF THE DRAWINGS

FIG. 1 shows a schematic representation of a nucleic acid amplification method according to the present invention.

FIG. 2 shows a direct comparison of Inosine and Xanthosine containing oligonucleotides using conditions optimised for Inosine containing primers. A. Amplification of the target template using conditions optimised for inosine-containing oligonucleotides. B. Amplification of the target template using conditions more optimised for Xanthosine.

FIG. 3 shows results of isothermal amplification using the Bst polymerase/TMA endonuclease system.

FIG. 4 shows results of isothermal amplification using the Bst polymerase/TMA endonuclease system with the addition of NaCl. A. Inosine containing oligonucleotide control. B. Xanthosine containing oligonucleotide +50 mM NaCl. C. Xanthosine containing oligonucleotide +100 mM NaCl.

FIG. 5 shows results of real time comparisons of Xanthosine and Inosine containing amplification primers

### MODE(S) FOR CARRYING OUT THE INVENTION

### Materials and Methods

### Primers

Primers can be synthesised using any commercially available DNA synthesis service or in-house DNA synthesisers. Xanthosine can be incorporated into the primer at any position using standard phosphoamidite synthesis technology.

### Enzymes

The enzyme that recognises Xanthosine in double-stranded DNA and causes a nick or excises a base in one DNA strand at or near the Xanthosine is preferably Endonuclease V (deoxyinosine 3′ endonuclease) (NEB catalogue number M0305S) or the thermostable version of endonuclease V (TMA endonuclease V) from T. maritima (Fermentas catalogue number EN0141). It will be appreciated, however, that modified or variant forms of Endonuclease V or enzymes having the functional characteristics of Endonuclease V would also be suitable

Enzymes capable of strand displacement include Klenow exo-, Bst DNA polymerase large fragment, Bca polymerase, Vent exo, Deep Vent exo-, M-MuLV reverse transcriptase, 9° Nm DNA polymerase and Phi29 DNA polymerase.

The DNA polymerase can be any suitable polymerase having strand displacement capability. Examples include, but not limited to, Klenow exo- (New England Biolabs (NEB) catalogue number M0212S), Bst DNA polymerase large fragment (NEB catalogue number M0275S), Vent exo- (NEB catalogue number M0257S), Deep Vent exo- (NEB catalogue number M0259S), M-MuLV reverse transcriptase (NEB catalogue number M0253S), 9° Nm DNA polymerase (NEB catalogue number M0260S) and Phi29 DNA polymerase (NEB catalogue number M0269S) ThermoPhi™ (Prokaria ehf), Tfi polymerase (Invitrogen) and Bca polymerase (Takara). Preferably, the DNA polymerase is Klenow Exo- or Bst polymerase.

### Amplification

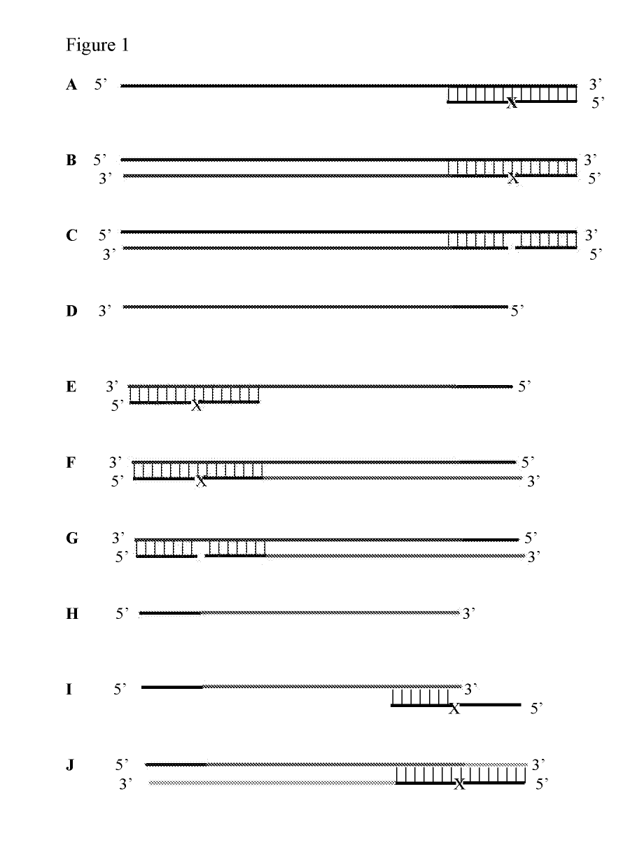

Amplification according to the present invention occurs in the following manner (see FIG. 1):

the first primer binds to one strand of DNA (A),

the DNA polymerase extends the first primer forming a double stranded molecule having a first newly synthesised strand containing X Xanthosine (B),

the nicking enzyme causes a nick or base excision at or near Xanthosine of the extended DNA (C),

the strand displacing enzyme or DNA polymerase capable of strand displacement displaces the first newly synthesised strand (D),

the second primer binds to the displaced first newly synthesised strand (E),

the DNA polymerase extends the second primer forming a double stranded molecule having a second newly synthesised strand containing Xanthosine (F),

the nicking enzyme causes a nick or base excision at or near Xanthosine of the extended DNA (G),

the strand displacing enzyme or DNA polymerase capable of strand displacement displaces the second newly synthesised strand (H),

the first primer binds to the displaced second newly synthesised strand (I), and

the process continues forming repeated newly synthesised strands of DNA (J).

The polymerase should copy the first primer in a 5′-3′ direction as if this does not occur the reaction would stop after the third cycle of amplification as the nick site will be lost preventing further amplification. The above reaction will then continue cycling with repeated rounds of nicking, extension and displacement. The primer is usually regenerated by the polymerase to allow successive rounds of amplification.

### Results

Direct Comparison of Inosine and Xanthosine Containing Oligonucleotides Using Conditions Optimised for Inosine Containing Primers

Unmethylated Target Oligo
(bisulphite treated equivalent)
(SEQ ID NO: 1)
AGGGAATTTTTTTTTGTGATGTTTTGGTGIGTTAGTTTGTTGIGTATATT
TTGTTGTGGTTTTTTTTTTGGTTTTTTTGGTTAGTTGTGTGGTGATTTTG
GGGATTTTAG
Inosine Unmet-F
(SEQ ID NO: 2)
AGGGAATTTTTTTTTGTGITGTTTTGGTGTGTTAGTTTG
I = Inosine
Inosine Unmet-R
(SEQ ID NO: 3)
AAAAAAACCAATCAACICACCACTAAAACCCCTAAAATC
I = Inosine
Xanthosine Unmet-F
(SEQ ID NO: 4)
AGGGAATTTTTTTTTGTGXTGTTTTGGTGTGTTAGTTTG
X = Xanthosine
Xanthosine Unmet-R
(SEQ ID NO: 5)
AAAAAAACCAATCAACXCACCACTAAAACCCCTAAAATC
X = Xanthosine

### A Klenow/Endonuclease V Reaction Conditions

Optimised conditions for oligonucleotides containing inosine

| | | |
|---|---|---|
| X10 Stoffel buffer | 1 | μl |
| 10 mM dNTPs | 0.5 | μl |
| 100 ng/μl Primer 1 | 0.5 | μl |
| 100 ng/μl Primer 2 | 0.5 | μl |
| Endonuclease V | 0.05 | μl |
| Klenow exo- | 0.4 | μl |
| 25 mM MgSO₄ | 0.4 | μl |
| Water | 5.55 | μl |

One μl of serially diluted target DNA was added to each reaction and the samples incubated at 42° C. for 4 hours.

### B Optimisation of Conditions for Xanthosine Conditions for Oligonucleotides Containing Inosine

| Mix A | | | Mix B | | |
|---|---|---|---|---|---|
| X10 Stoffel buffer | 0.5 | μl | X10 Stoffel buffer | 0.5 | μl |
| 10 mM dNTPs | 0.5 | μl | 100 ng/μl Primer 2 | 0.125 | μl |
| 100 ng/μl Primer 2 | 0.125 | μl | Endonuclease V | 0.05 | μl |
| 25 mM MgCl₂ | 0.5 | μl | Klenow exo- | 0.4 | μl |
| Water | 3.4 | μl | T4gp32 | 0.5 | μl |
| | | | Water | 3.42 | μl |

One μl of serially diluted target DNA was added to mixture A and the samples heated to 94° C. for 2 minutes then cooled to 42° C. and mixture B added then the samples were incubated at 42° C. for 4 hours.

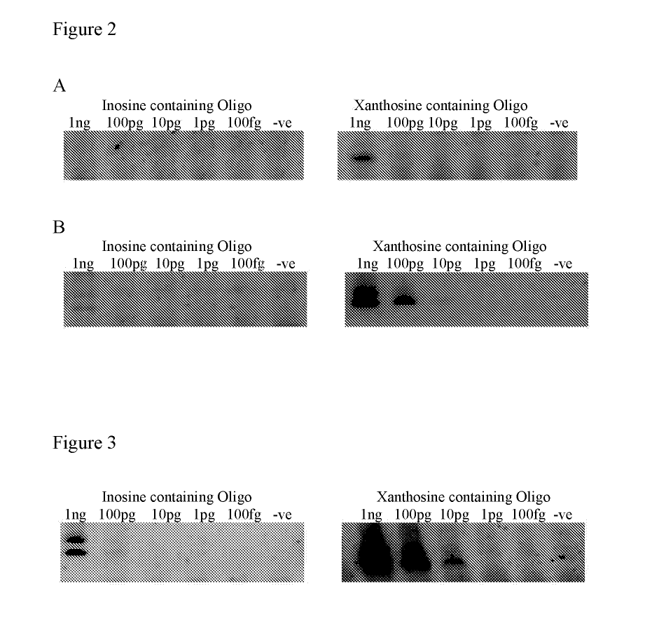

FIG. 2A shows isothermal amplification of the target template using conditions optimised for inosine-containing oligonucleotides. As can be seen from the results, 1 ng of target template is detected using the Xanthosine containing oligonucleotides but not with the inosine oligonucleotides, demonstrating that the Xanthosine modification works more efficiently in an isothermal amplification reaction compared to the inosine modification.

FIG. 2B shows conditions more optimised for Xanthosine containing oligonucleotides in which signals can be detected with as little as 10 pg of starting template compared to 1 ng with the inosine primers. This represents a 100 fold increase in amplification when using the Xanthosine modified primers. The primer concentration had to be reduced presumably due to the fact that the Xanthosine oligonucleotides are more resistant to nicking compared to the inosine modification.

Isothermal Amplification Using the Bst Polymerase/TMA Endonuclease System

| Mix A | | | Mix B | | |
|---|---|---|---|---|---|
| X10 Thermopol buffer | 0.5 | μl | X10 Thermopol buffer | 0.5 | μl |
| 10 mM dNTPs | 0.25 | μl | 100 ng/μl Primer 2 | 0.125 | μl |
| 100 ng/μl Primer2 | 0.125 | μl | TMA Endonuclease V | 0.1 | μl |
| T4gp32 | 0.5 | μl | Bst polymerase | 0.5 | μl |
| 100 mM DTT | 0.5 | μl | Water | 3.8 | μl |
| Water | 3.12 | μl | | | |

One μl of serially diluted target DNA was added to mixture A and the samples heated to 94° C. for 2 minutes then cooled to 45° C. for 2 minutes then heated to 60° C. for 2 minutes and mixture B added then the samples were incubated at 60° C. for 45 minutes.

FIG. 3 shows isothermal amplification of the target template using conditions optimised for Xanthosine-containing oligonucleotides using the heat stable Bst polymerase/TMA Endonuclease V amplification combination. As can be seen from the results, 10 pg of target template is easily detected using the Xanthosine containing oligonucleotides but not with the inosine oligonucleotides. In addition, this reaction can be carried out in as little as 45 minutes compared to the 4-hour incubation time required using the Klenow-exo-/Endonuclease V system.

Isothermal Amplification Using the Bst Polymerase/TMA Endonuclease System with the Addition of NaCl

| Mix A | | | Mix B | | |
|---|---|---|---|---|---|
| X10 Thermopol | 0.5 | μl | X10 Thermopol buffer | 0.5 | μl |
| buffer | | | 100 ng/μl Primer 2 | 0.125 | μl |
| 10 mM dNTPs | 0.25 | μl | TMA Endonuclease V | 0.1 | μl |
| 100 ng/μl | 0.125 | μl | Bst polymerase | 0.5 | μl |
| Primer 2 | | | Water | 3.8 | μl |
| 1M NaCl | 0.5/1 | μl | | | |
| 100 mM DTT | 0.5 | μl | | | |
| Water | 3.12/2.63 | μl | | | |

One μl of serially diluted target DNA was added to mixture A and the samples heated to 94° C. for 2 minutes then cooled to 45° C. for 2 minutes then heated to 60° C. for 2 minutes and mixture B added then the samples were incubated at 60° C. for 45 minutes.

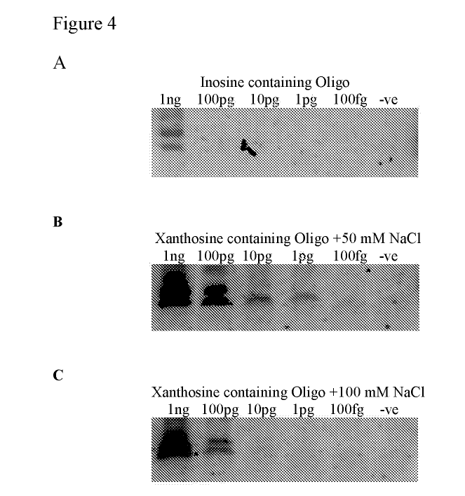

FIG. 4 demonstrates that the addition of salt can improve the amplification efficiency using Xanthosine-containing primers. However, from the results it would seem that increasing the salt concentration above 50 mM in the final reaction could lead to loss of signal. Using this system a 1000 fold increase in sensitivity can be seen when using the Xanthosine-containing primers compared to primers modified with inosine.

### Real Time Comparisons of Xanthosine and Inosine Containing Amplification Primers

Methylated Target Oligo
(bisulphite treated equivalent) at 1 μg/μl
(SEQ ID NO: 6)
AGGGAATTTTTTTTCGCGATGTTTCGGCGCGTTAGTTCGTTGCGTATATT
TCGTTGCGGTTTTTTTTTTGGTTTTTTCGGTTAGTTGCGCGGCGATTTCG
GGGATTTTAG
Inosine Unmet-F
(SEQ ID NO: 7)
AGGGAATTTTTTTTCGCIATGTTTCGGCGCGTTAGTTCGT
I = Inosine
Inosine Unmet-R2
(SEQ ID NO: 8)
CTAAAATCCCCGAAATCGCCGCICAACTAACCGAAAAAAC
I = Inosine
Xanthosine Unmet-F
(SEQ ID NO: 9)
AGGGAATTTTTTTTCGCXATGTTTCGGCGCGTTAGTTCGT
X = Xanthosine
Xanthosine Unmet-R
CTAAAATCCCCGAAATCGCCGCXCAACTAACCGAAAAAAC
(SEQ ID NO: 10)
X = Xanthosine
Molecular Beacon probe
(SEQ ID NO: 11)
[6FAM] CGATGCGCGTATATTTCGTTGCGGTTTTTTGCATCG
[DABC]

### Reaction Mixes

| Mix A | | | Mix B | | |
|---|---|---|---|---|---|
| X10 Thermopol buffer | 0.5 | μl | X10 Thermopol buffer | 0.5 | μl |
| 10 mM dNTPs | 0.25 | μl | 100 ng/μl Primer F | 0.25 | μl |
| 100 ng/μl Primer R | 0.25 | μl | TMA Endonuclease V | 0.1 | μl |
| 5M Betaine | 1.0 | μl | Bst polymerase | 1.0 | μl |
| Water | 2.75 | μl | Water | 2.65 | μl |
| | | | 20 μM probe | 0.5 | μl |

The methylated target oligo was serially diluted from 1/1000 to 1/10,000,000 and 1 μl of material added to reaction mix A for each sample to be tested. The tubes were heated to 95° C. for 1 minute, cooled to 45° C. for 1 minute then heated to 60° C. for 5 minutes then mix B added to each sample.

Samples were then cycled 60° C. for 5 minute, 45° C. for 10 seconds (plate read on Fam channel). 20 cycles were performed.

TABLE 1
Fluorescence Results

| Oligo added | | Well | Inosine (Ct) | Well | Xanthosine (Ct) |
|---|---|---|---|---|---|
| 1 | ng | A1 | 12.72 | A7 | 7.59 |
| 100 | pg | A2 | 13.73 | A8 | 8.05 |
| 10 | pg | A3 | 16.79 | A9 | 10.91 |
| 1 | pg | A4 | No Ct | A10 | 13.49 |
| 100 | fg | A5 | No Ct | A11 | 14.84 |

| | | | | |
|---|---|---|---|---|
| NTC | A6 | No Ct | A12 | No Ct |

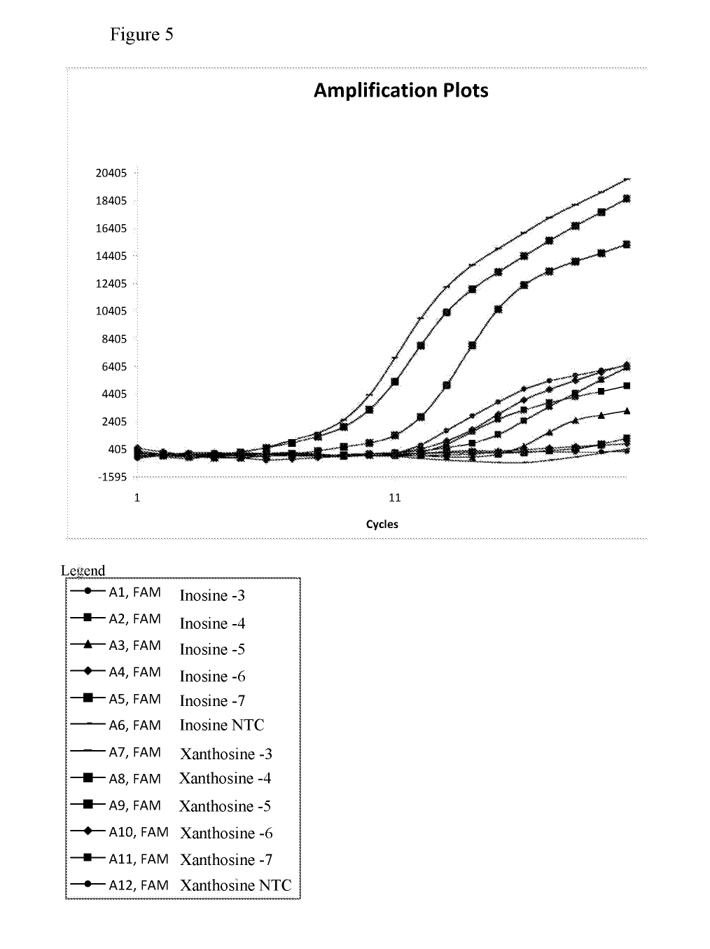

FIG. 5 shows real time isothermal amplification plots using both inosine and Xanthosine containing oligonucleotides. The data clearly shows that Xanthosine is a much-improved substrate for the endonuclease V reaction when compared with inosine. Amplification signals for Xanthosine are seen at the lowest level tested 1100 fg) whereas using inosine signals are only detected at the 10 pg level. In addition the Fluorescence signals generated using Xanthosine are over 3 times stronger than with inosine.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for isothermal DNA amplification comprising:
providing to DNA to be amplified an amplification mix comprising:
a first primer at least partially complementary to a region of DNA and containing Xanthosine,
a second primer at least partially complementary to a region of DNA and containing Xanthosine,
a DNA polymerase,
an enzyme capable of strand displacement,
an enzyme that recognises Xanthosine in double-stranded DNA and causes a nick or excises a base in one DNA strand at or near the Xanthosine; and
amplifying the DNA substantially without thermal cycling.

2. The method according to claim 1 wherein the DNA is denatured prior to, during, or after addition of the amplification mix.

3. The method according to claim 1 or 2 wherein the first primer is at least partially complementary to a region of a first strand of DNA, and the second primer is at least partially complementary to a region of DNA of the second strand of DNA.

4. The method according to any one of claims 1 to 3 wherein the first and second primers are oligonucleotides, oligonucleotide analogues, or oligonucleotides of chimeric nature.

5. The method according to claim 4 wherein the primers are deoxyoligonucleotides.

6. The method according to claim 4 wherein the primers are oligonucleotide analogues selected from the group consisting of intercalating nucleic acid (INA), peptide nucleic acid (PNA), hexitol nucleic acid (HNA), MNA, altritol nucleic acid (ANA), locked nucleic acid (LNA), cyclohexanyl nucleic acid (CAN), CeNA, TNA, (2′-NH)-TNA, nucleic acid based conjugates, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R—RNA, 2′-OR—RNA, α-L-RNA, β-D-RNA, mixtures thereof and hybrids thereof, and phosphorous atom modifications thereof.

7. The method according to claim 6 wherein the primers contain one or more intercalator pseudonucleotides.

8. The method according to any one of claims 1 to 7 wherein the primers can have two or more Xanthosines positioned close or spaced apart by at least several regular bases.

9. The method according to any one of claims 1 to 8 wherein the DNA polymerase is selected from the group consisting of Taq polymerase Stoffel fragment, Taq polymerase, Advantage DNA polymerase, AmpliTaq, Amplitaq Gold, Titanium Taq polymerase, KlenTaq DNA polymerase, Platinum Taq polymerase, Accuprime Taq polymerase, Pfu polymerase, Pfu polymerase turbo, Vent polymerase, Vent exo-polymerase, Pwo polymerase, 9° Nₘ DNA polymerase, Therminator, Pfx DNA polymerase, Expand DNA polymerase, rTth DNA polymerase, DyNAzyme™ EXT Polymerase, Klenow fragment, DNA polymerase 1, DNA polymerase, T7 polymerase, Sequenase™, T4 DNA polymerase, Bst polymerase, Bca polymerase, Tfi polymerase, phi-29 DNA polymerase, DNA polymerase Beta, and modified versions thereof.

10. The method according to any one of claims 1 to 9 wherein the strand displacement enzyme is selected from the group consisting of Helicases, AP endonucleases, and mismatch repair enzymes capable of stand displacement, or modified enzymes capable of stand displacement.

11. The method according to any one of claims 1 to 8 wherein the DNA polymerase also has strand displacement capability and is selected from the group consisting of Klenow exo-, Bst DNA polymerase large fragment, Bca polymerase, Vent exo-, Deep Vent exo-, M-MuLV reverse transcriptase, 9° Nm DNA polymerase, and Phi29 DNA polymerase.

12. The method according to claim 11 wherein the DNA polymerase is Klenow Exo- or Bst polymerase.

13. The method according to any one of claims 1 to 12 wherein the DNA polymerase is exonuclease deficient.

14. The method according to any one of claims 1 to 13 wherein the enzyme capable of recognising Xanthosine in double stranded DNA is Endonuclease V, hOGG1 or Fpg.

15. The method according to any one of claims 1 to 14 wherein the amplification mix further comprises additives required for DNA amplification including nucleotides, buffers, diluents, magnesium or manganese ions, single stranded binding proteins, and co-factors.

16. The method according to claim 15 wherein the single stranded binding proteins are T4gp32, RecA or SSB.

17. The method according to any one of claims 1 to 16 wherein amplification is carried out at a temperature from 20° C. to about 75° C.

18. The method according to claim 17 wherein the temperature is about 42° C. or 60° C.

19. The method according to any one of claims 1 to 18 wherein amplification is carried out in the presence of NaCl.

20. The method according claim 19 wherein NaCl concentration is up to about 100 mM.

21. A primer for isothermal DNA amplification containing at least one internal Xanthosine and when bound to a region of DNA forms a site recognised by an enzyme capable of causing a nick or excising a base in one DNA strand at or near the site of the Xanthosine.

22. Use of a primer according to claim 21 for DNA amplification substantially without thermal cycling.


SEQUENCE LISTING (11 sequences)

SEQ ID NO: 1 - DNA, 110 bp, Artificial Sequence
  Oligonucleotide primer
agggaatttttttttgtgatgttttggtgtgttagtttgttgtgtatattttgttgtggttttttttttggtttttttggttagttgtgtggtgattttggggattttag

SEQ ID NO: 2 - DNA, 40 bp, Artificial Sequence
  Oligonucleotide primer
ctaaaatccccgaaatcgccgcncaactaaccgaaaaaac

SEQ ID NO: 3 - DNA, 39 bp, Artificial Sequence
  Oligonucleotide primer
aaaaaaaccaatcaacncaccactaaaacccctaaaatc

SEQ ID NO: 4 - DNA, 39 bp, Artificial Sequence
  Oligonucleotide primer
agggaatttttttttgtgntgttttggtgtgttagtttg

SEQ ID NO: 5 - DNA, 39 bp, Artificial Sequence; identical to SEQ ID NO: 3
  Oligonucleotide primer

SEQ ID NO: 6 - DNA, 110 bp, Artificial Sequence
  Oligonucleotide primer
agggaattttttttcgcgatgtttcggcgcgttagttcgttgcgtatatttcgttgcggttttttttttggttttttcggttagttgcgcggcgatttcggggattttag

SEQ ID NO: 7 - DNA, 40 bp, Artificial Sequence
  Oligonucleotide primer
agggaattttttttcgcnatgtttcggcgcgttagttcgt

SEQ ID NO: 8 - DNA, 40 bp, Artificial Sequence; identical to SEQ ID NO: 2
  Oligonucleotide primer

SEQ ID NO: 9 - DNA, 40 bp, Artificial Sequence; identical to SEQ ID NO: 7
  Oligonucleotide primer

SEQ ID NO: 10 - DNA, 40 bp, Artificial Sequence; identical to SEQ ID NO: 2
  Oligonucleotide primer

SEQ ID NO: 11 - DNA, 36 bp, Artificial Sequence
  Oligonucleotide primer
cgatgcgcgtatatttcgttgcggttttttgcatcg